(19) United States
(12) Patent Application Publication
(10) Pub. No.: US 2019/0262257 A1
(43) Pub. Date: Aug. 29, 2019

(54) MATTE LIP COMPOSITIONS

(71) Applicant: L'OREAL, Paris (FR)

(72) Inventors: Roselin ROSARIO-MELÉNDEZ, New York, NY (US); Susan Ashley DeSTENO, Springfield, NJ (US); Chao ZHU, Berkeley Heights, NJ (US)

(73) Assignee: L'OREAL, Paris (FR)

(21) Appl. No.: 15/908,646

(22) Filed: Feb. 28, 2018

(51) Int. Cl. A61K 8/891; A61K 8/898; A61K 8/25; A61K 8/88; A61K 8/81; A61Q 1/04

(52) U.S. Cl. CPC A61K 8/891; A61K 8/898; A61Q 1/04; A61K 8/88; A61K 8/8111; A61K 8/25

(57) ABSTRACT

The present invention relates to matte lip compositions including at least one siloxysilicate resin, at least one polyorganosiloxane copolymer, and at least one lipophilic silica, and preferably including at least one non-lipophilic filler and at least one non-volatile hydrocarbon oil, as well as to methods of making and applying such compositions to lips.

### FIELD OF THE INVENTION

The present invention relates to matte lip compositions comprising at least one siloxysilicate resin, at least one polyorganosiloxane copolymer, at least one lipophilic silica, at least one non-lipophilic filler and at least one non-volatile hydrocarbon oil, as well as to methods of making such compositions and methods of applying such compositions to lips.

### DISCUSSION OF THE BACKGROUND

Many cosmetic compositions, including pigmented cosmetics such as lipsticks or lip colors, have been formulated in an attempt to possess long-wearing properties upon application. Unfortunately, many of these compositions do not generally possess good long-wear/transfer-resistance properties as well as good appearance properties (for example, matte properties) and/or application properties.

With respect to lip compositions, commercial products containing silicon resins such as MQ resins are known. Such products are known to provide good long wear properties/transfer-resistance. However, such products possess poor application properties and/or poor feel upon application (for example, feels rough).

For example, U.S. Pat. Nos. 8,080,257, 7,758,848, U.S. application 2004/0170586, PCT application WO 2003/105801, and PCT application WO 2010/105952, disclose compositions containing nylon-611/dimethicone and MQ resins in broad, general amounts.

Typically, a second composition (topcoat) is separately applied to such products to improve poor properties of the compositions to make the products acceptable to consumers. However, topcoat compositions tend to decrease the long-wear/transfer-resistance properties of the lip compositions, thereby rendering the long-wear/transfer resistant composition less acceptable to consumers and less acceptable for their intended purpose.

Thus, there remains a need for improved matte lip compositions having improved cosmetic properties, particularly good transfer-resistance, feel and/or matte characteristics upon application.

### SUMMARY OF THE INVENTION

The present invention relates to matte lip compositions comprising at least one siloxysilicate resin in an amount of from about 7% to about 15% by weight, at least one polyorganosiloxane copolymer in an amount of from about 4.5% to about 10% by weight, at least one lipophilic silica in an amount of from about 0.5% to about 1.5% by weight, at least one non-lipophilic filler in an amount of from about 0% to about 3.5% by weight, and at least one non-volatile hydrocarbon oil in an amount of from about 0% to about 3.5% by weight, all weights being based on the total weight of the composition. Preferably, the composition further comprises at least one colorant and at least one volatile solvent.

The present invention also relates to matte lip compositions comprising at least one siloxysilicate resin in an amount of from about 10% to about 14% by weight, at least one polyorganosiloxane copolymer in an amount of from about 6.5% to about 10% by weight, at least one lipophilic silica in an amount of from about 1% to about 1.5% by weight, at least one non-lipophilic filler in an amount of from about 1% to about 2% by weight, and at least one non-volatile hydrocarbon oil in an amount of from about 1% to about 2% by weight, all weights being based on the total weight of the composition. Preferably, the composition further comprises at least one colorant and at least one volatile solvent.

The present invention also relates to methods of making a matte lip composition comprising combining at least one siloxysilicate resin in an amount of from about 7% to about 15% by weight, at least one polyorganosiloxane copolymer in an amount of from about 4.5% to about 10% by weight, at least one lipophilic silica in an amount of from about 0.5% to about 1.5% by weight, at least one non-lipophilic filler in an amount of from about 0% to about 3.5% by weight, and at least one non-volatile hydrocarbon oil in an amount of from about 0% to about 3.5% by weight, all weights being based on the total weight of the composition, during preparation of the lip composition to form a matte lip composition.

The present invention also relates to methods of making a matte lip composition comprising combining at least one siloxysilicate resin in an amount of from about 10% to about 14% by weight, at least one polyorganosiloxane copolymer in an amount of from about 6.5% to about 10% by weight, at least one lipophilic silica in an amount of from about 1% to about 1.5% by weight, at least one non-lipophilic filler in an amount of from about 1% to about 2% by weight, and at least one non-volatile hydrocarbon oil in an amount of from about 1% to about 2% by weight, all weights being based on the total weight of the composition, during preparation of the lip composition to form a matte lip composition.

The present invention further relates to methods for making up and/or protecting lips comprising applying to the lips a matte lip composition comprising at least one siloxysilicate resin in an amount of from about 7% to about 15% by weight, at least one polyorganosiloxane copolymer in an amount of from about 4.5% to about 10% by weight, at least one lipophilic silica in an amount of from about 0.5% to about 1.5% by weight, at least one non-lipophilic filler in an amount of from about 0% to about 3.5% by weight, and at least one non-volatile hydrocarbon oil in an amount of from about 0% to about 3.5% by weight, all weights being based on the total weight of the composition.

The present invention further relates to methods for making up and/or protecting lips comprising applying to the lips a matte lip composition comprising at least one siloxysilicate resin in an amount of from about 10% to about 14% by weight, at least one polyorganosiloxane copolymer in an amount of from about 6.5% to about 10% by weight, at least one lipophilic silica in an amount of from about 1% to about 1.5% by weight, at least one non-lipophilic filler in an amount of from about 1% to about 2% by weight, and at least one non-volatile hydrocarbon oil in an amount of from about 1% to about 2% by weight, all weights being based on the total weight of the composition.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to lips and evaluating the color of the composition after an extended period of time. For example, the color of a composition may be evaluated immediately following application to lips and these characteristics may then be re-evaluated and compared after a certain amount of time. Further, these characteristics may be evaluated with respect to other compositions, such as commercially available compositions.

Anhydrous refers to a composition not containing any water, that is to say a composition in which the water that may be present comes only from the water of crystallization or of adsorption of the starting materials. In any case, an “anhydrous composition” in accordance with the present invention contains less than 3% by weight of water, preferably less than 1% by weight, and better still less than 0.5% by weight of water, relative to the total weight of the composition. A composition of the present invention may also contain no water.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions. For lip compositions, “long wear” typically means the composition remains on the lips at least about 4 hours up to about 24 hours, and retains rich color even after eating.

“Liquid” or “liquid cosmetic” or “liquid lipstick” or “liquid composition” means a composition having a fixed volume, flows to cover the bottom and assumes the shape of the portion of the container it fills and is slightly compressible (as disclosed in General chemistry, Fourth Edition 2005, p. 434).

“Adhesion” as used herein, refers to chemical or physical bonding between a coating and a substrate. Good adhesion between a lip composition and lips should translate to good wear properties on consumers. Adhesion properties can be quantified by in-vitro method such as a cross-cut adhesion test. In the test, a lattice pattern is cut into the coating and penetrates through to the substrate. A pressure sensitive tape is applied to the sample and then pulled off. The adhesion property can be quantified by the area of the coating remaining after peeling. For example, if the whole film remains after peeling, it indicates excellent adhesion. If most of the film gets peeled off, it indicates poor adhesion. The cross-cut test is an industrial standard test for testing adhesion for coatings. (Reference # ISO/DIN 2409, ASTM D3359).

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

“Matte” in compositions as used herein refers to compositions having little to no light reflection. For example, matte compositions can have average gloss properties, measured at 60°, of less than or equal to 10, for example 9, preferably less than or equal to 8, 6, 5, 4 or 1, including all ranges and subranges therebetween such as 1-10, 1-5, 2-10, 3-8, less than 1, less than 3, less than 5, etc.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of phenylated solvents” means that non-volatile solvents are preferably omitted (that is 0% by weight), but can be present in the composition at an amount of less than about 0.25% by weight, typically less than about 0.1% by weight, typically less than about 0.05% by weight, based on the total weight of the composition as a whole.

The compositions and methods of the present invention can “comprise,” “consist of” or “consist essentially of” the essential elements and limitations of the invention described herein, as well as any additional or optional ingredients, components, or limitations described herein or otherwise useful. For purposes of the compositions and methods of the present invention where the invention “consists essentially of” the identified ingredients and/or process steps, the sole “basic and novel property” of such compositions and/or methods is “matte.” Further, given that it is contemplated that other mattifying agents can be added to the invention methods and compositions in the context of the present invention, a “material effect” on the basic and novel property of the invention can only be an adverse effect. That is, because positive effects on matte properties (such as those effected by mattifying agents such as silica) are within the scope of the present invention, only ingredients which have a material adverse effect on matte properties of the invention compositions would be relevant to determining whether or not compositions or methods “consist essentially of” the required elements.

The composition of the present invention may be in any form, either liquid or non-liquid (semi-solid, soft solid, solid, etc.). For example, it may be a paste, a solid, a gel, or a cream. It may be an emulsion, such as an oil-in-water or water-in-oil emulsion, a multiple emulsion, such as an oil-in-water-in-oil emulsion or a water-in-oil-in-water emulsion, or a solid, rigid or supple gel. The composition of the invention may, for example, comprise an external or continuous fatty phase. The composition can also be a molded composition or cast as a stick or a dish.

Matte Lip Composition

According to the present invention, a matte lip composition comprising at least one siloxysilicate resin in an amount of from about 7% to about 15% by weight, at least one polyorganosiloxane copolymer in an amount of from about 4.5% to about 10% by weight, at least one lipophilic silica in an amount of from about 0.5% to about 1.5% by weight, at least one non-lipophilic filler in an amount of from about 0% to about 3.5% by weight, and at least one non-volatile hydrocarbon oil in an amount of from about 0% to about 3.5% by weight, all weights being based on the total weight of the composition is provided.

Siloxysilicate Resin

In accordance with the present invention, matte lip compositions comprising at least one siloxysilicate resin are provided.

One non-limiting example of a siloxysilicate in accordance with the present invention is trimethylsiloxysilicate, which may be represented by the following formula:

[(CH₃)₃SiO]ₓ(SiO_{4/2})_{y}

wherein x and y may, for example, range from 50 to 80. Such siloxysilicates are commercially available from General Electric, Dow Corning, Wacker, Milliken, Siltech, Grant Industries, Momentive and Shin-Etsu Silicones under the tradename Resin MQ®.

The at least one siloxysilicate resin is preferably present in the matte lip compositions of the present invention in an amount ranging from about 7% to about 15% by weight, and preferably from about 10% to about 14% by weight, including all ranges and subranges therebetween, all weights being based on the weight of the composition as a whole.

Polyorganosiloxane Copolymer

In accordance with the present invention, matte lip compositions comprising at least one polyorganosiloxane copolymer are provided. The polyorganosiloxane copolymer useful herein is preferably a polymer (homopolymer or copolymer) having at least one moiety which contains: at least one polyorganosiloxane group consisting of 1 to about 1000 organosiloxane units in the chain of the moiety or in the form of a graft, and at least two groups capable of establishing hydrogen interactions. Non-limiting examples of polyorganosiloxane copolymers are disclosed, for example in U.S. Pat. No. 8,945,525, the disclosure of which is hereby incorporated by reference in its entirety.

Additional polyorganosiloxane copolymers which may be used in the long-wear lip compositions of the present invention include those described in documents U.S. Pat. Nos. 5,874,069, 5,919,441, 6,051,216, and 5,981,680, the entire contents of which are hereby incorporated by reference in their entirety.

A preferred polyorganosiloxane copolymer for use in the present invention contain at least one moiety chosen from formula (III):

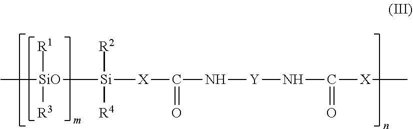

and formula (IV)

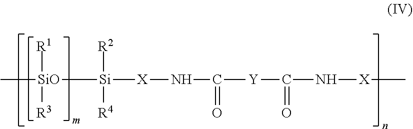

in which:

(a) R¹, R², R³ and R⁴ are the same or different and may be selected from the group consisting of methyl, ethyl, propyl, isopropyl, a siloxane chain, and phenyl;

(b) X is a linear or branched chain alkylene having 1-30 carbons;

(c) Y is selected from the group consisting of linear or branched chain alkylenes having 1-40 carbons;

(d) m is a number between 1 and 700;

(e) n is a number between 1 and 500.

Particularly preferred polyorganosiloxane copolymers useful herein are commercially available from Dow Corning under the tradenames DC 8178® and DC 8179®, which are known under the INCI denomination of Nylon-611/Dimethicone Copolymer.

Preferably, the polyorganosiloxane copolymer is present in the matte lip compositions of the present invention in an amount ranging from about 4.5% to about 10% by weight, and preferably from about 6.5% to about 10% by weight, including all ranges and subranges therebetween, all weights being based on the weight of the composition as a whole.

Lipophilic Silica

In accordance with the present invention, matte lip compositions comprising at least one lipophilic silica are provided. The term “lipophilic silica” means a silica that is capable of swelling in a lipophilic medium.

Suitable lipophilic silicas include, but are not limited to, pyrogenic silica optionally with hydrophobic surface treatment whose particle size is less than 1 micron, preferably less than 500 nm, preferably less than 100 nm, preferably from 5 nm to 30 nm, including all ranges and subranges therebetween. It is in fact possible to modify the surface of silica chemically, by a chemical reaction producing a decrease in the number of silanol groups present on the surface of the silica. The silanol groups can notably be replaced with hydrophobic groups: a lipophilic silica is then obtained. The hydrophobic groups can be, for example:

trimethylsiloxyl groups, which are notably obtained by treatment of pyrogenic silica in the presence of hexamethyldisilazane. Silicas treated in this way are called “Silica silylate” according to the CTFA (6th edition, 1995). They are for example marketed under the references “AEROSIL R812®” by the company Degussa, “CAB-O-SIL TS-530®” by the company Cabot; and/or

dimethylsilyloxyl or polydimethylsiloxane groups, which are notably obtained by treatment of pyrogenic silica in the presence of polydimethylsiloxane or dimethyldichlorosilane. Silicas treated in this way are called “Silica dimethyl silylate” according to the CTFA (6th edition, 1995). They are for example marketed under the references “AEROSIL R972®”, “AEROSIL R974®” by the company Degussa, “CAB-O-SIL TS-610®”, “CAB-0-SIL TS-720®” by the company Cabot.

Preferably, the lipophilic silica is present in the matte lip compositions of the present invention in amounts of active material generally ranging from about 0.5% to about 1.5%, and preferably from about 1% to about 1.5%, by weight, based on the total weight of the composition, including all ranges and subranges in between.

Non-Lipophilic Filler

In accordance with preferred embodiments of the present invention, matte lip compositions further comprising at least one non-lipophilic filler are provided. However, compositions of the present invention may be free of non-lipophilic filler.

The non-lipophilic filler may be one commonly used in the art in cosmetic compositions. The fillers may be lamellar or non-lamellar, inorganic or organic particles. Representative, non-limiting examples of such fillers include mica, silica, kaolin, polyamide powders, polyalanine powders, polyethylene powders, and tetrafluoroethylene polymer powders, for instance polytetrafluoroethylene (Teflon®).

According to preferred embodiments, the non-lipophilic filler is at least one polyamide powder. The polyamide powders useful in the present invention may have particles of the same or different shapes, for example spheres, full or hollow, needles or platelets, etc. Preferably, however, the particles have a shape which is approximately spherical. Suitable examples of commercially available polyamide powders include those sold under the name Nylon® or Orgasol® powders by Arkema.

Preferably, the polyamide particles have an average particle size ranging from 1 to 100 microns, preferably 3 to 80 microns, and preferably 5 to 60 microns, including all ranges and subranges therebetween.

Preferably, the polyamide particles have a specific surface area ranging from 1 to 20 m²/g, preferably from 1 to 9 m²/g, and preferably from 1 to 6 m²/g, including all ranges and subranges therebetween.

Preferably, the polyamide particles have a density ranging from 1.00 to 1.20 g/cm³, and preferably from 1.03 to 1.15 g/cm³, including all ranges and subranges therebetween.

Preferably, the polyamide particles have a melting point ranging from 140° C. to 220° C., and preferably from 175° C. to 220° C., including all ranges and subranges therebetween.

Preferably, the non-lipophilic filler is present in the matte lip compositions of the present invention in amounts of active material generally ranging from greater than 0% to about 3.5%, and preferably from about 1% to about 2%, by weight, based on the total weight of the composition, including all ranges and subranges in between.

Non-Volatile Hydrocarbon Oil

In accordance with preferred embodiments of the present invention, matte lip compositions further comprising at least one non-volatile hydrocarbon oil are provided. However, compositions of the present invention may be free of non-volatile hydrocarbon oil.

According to preferred embodiments, the non-volatile hydrocarbon oil is apolar and/or is chosen from linear or branched hydrocarbons of mineral or synthetic origin.

Specific examples of suitable non-volatile hydrocarbon oils include, but are not limited to, liquid paraffin, liquid petroleum jelly, naphthalene oil, polybutylenes such as Indopol H-100 (molar mass or MW=965 g/mol), Indopol H-300 (MW=1340 g/mol) and Indopol H-1500 (MW=2160 g/mol) sold or manufactured by the company Amoco, hydrogenated polyisobutylenes such as Parleam® or Panalane H-300 E sold or manufactured by the company Amoco (MW=1340 g/mol), Viseal 20000 sold or manufactured by the company Synteal (MW=6000 g/mol) and Rewopal PIB 1000 sold or manufactured by the company Witco (MW=1000 g/mol), decene/butene copolymers, polybutene/polyisobutene copolymers, especially Indopol L-14, polydecenes and hydrogenated polydecenes such as Puresyn 10 (MW=723 g/mol) and Puresyn 150 (MW=9200 g/mol) sold or manufactured by the company Mobil Chemicals, and mixtures thereof.

Preferably, the non-volatile hydrocarbon oil is a “C3-C5 polyalkene” which includes all isomers of the relevant compounds. So, for example, “C4 polyalkene” includes polybutene and polyisobutene. It is to be further understood that the phrase “C3-C5 polyalkene” includes hydrogenated compounds and non-hydrogenated compounds.

Suitable examples of commercially-available C3-C5 polyalkenes include those sold under the Indopol® (non-hydrogenated compounds) and Panalane® (hydrogenated compounds) names by Ineos as noted above.

Preferably, hydrogenated C3-C5 polyalkenes have a molecular weight of 250 g/mol to 1500 g/mol, preferably from 300 g/mol to 1400 g/mol, and preferably from 350 g/mol to 1300 g/mol, including all ranges and subranges therebetween.

Preferably, non-hydrogenated C3-C5 polyalkenes have a molecular weight of 750 g/mol to 7500 g/mol, preferably from 900 g/mol to 6000 g/mol, and preferably from 1300 g/mol to 2500 g/mol, including all ranges and subranges therebetween.

Preferably, the non-volatile hydrocarbon oil is present in the matte lip compositions of the present invention in amounts of active material generally ranging from greater than 0% to about 3.5%, preferably from about 1% to about 2% and preferably from about 2% to about 3.5%, by weight, based on the total weight of the composition, including all ranges and subranges in between.

According to preferred embodiments, the weight ratio of siloxysilicate resin to polyorganosiloxane is from 3:1 to 1:1, preferably 2:1 to 1:1, and preferably 1.5:1 to 1:1, including all ranges and subranges therebetween.

According to preferred embodiments, the weight ratio of non-volatile hydrocarbon oil to lipophilic silica is from 5:1 to 1:1, preferably 4:1 to 1.5:1, and preferably 3:1 to 2:1, including all ranges and subranges therebetween.

According to preferred embodiments, the weight ratio of non-lipophilic filler to non-volatile hydrocarbon oil is from 2:1 to 1:1, preferably 1.5:1 to 1:1, and preferably 1.25:1 to 1:1, including all ranges and subranges therebetween.

Additional Ingredients

Volatile Solvent

The matte lip compositions of the present invention may optionally further comprise at least one volatile solvent.

Examples of suitable volatile solvents include volatile hydrocarbon-based oils such as, for example, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate, alcohols, and their mixtures. Preferably, the volatile hydrocarbon-based oils have a flash point of at least 40° C.

Examples of volatile hydrocarbon-based oils include, but are not limited to those given in Table 1 below.

TABLE 1

| Compound | Flash Point (° C.) |
|---|---|
| Isododecane | 43 |
| Isohexadecane | 102 |
| Isodecyl neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatile solvent may also be chosen from volatile silicone oils, which may be linear or cyclic, having a viscosity, at room temperature, of less than or equal to 6 cSt, and having from 2 to 7 silicon atoms, optionally substituted with alkyl or alkoxy groups of 1 to 10 carbon atoms.

Examples of suitable volatile silicone oils include, but are not limited to, those listed in Table 2 below.

TABLE 2

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3St) from Dow Corning | 102 | 3 |

The at least one volatile solvent, if present, is generally present in the compositions of the present invention in an amount ranging from about 5% to about 70% by weight; preferably from about 15% to about 65% by weight; and preferably from about 30% to about 60% by weight, including all ranges and subranges therebetween, all weights being based on the weight of the composition as a whole.

Non-Volatile Solvent

The matte lip compositions of the present invention may optionally further comprise at least one non-volatile solvent (oil).

Non-volatile oils include low viscosity oils (having a viscosity from about 5 to about 10 centipoise) and high viscosity oils (having a viscosity of from about 100 to about 10,000 centipoise), and mixtures thereof. In contrast to waxes, oils are liquids at room temperature.

Non-limiting examples of suitable non-volatile silicone oils include polymethylsiloxanes (PDMS) with a linear or cyclic silicone chain, which are liquid or pasty at room temperature, especially cyclopolydimethylsiloxanes (cyclomethicones) such as cyclohexasiloxane; polydimethyl-siloxanes (CTFA designation “dimethicones”) comprising alkyl or alkoxy groups, which are pendent or at the end of a silicone chain, these groups containing from 2 to 24 carbon atoms; polydiethyl siloxanes; and dimethicone fluids having viscosity from about 300 cPs at 25° C. to about 1500 cPs at 25° C. Particularly useful dimethicone fluids have viscosity from about 350 cPs at 25° C. to about 1000 cPs at 25° C.

Specific examples of suitable for this invention high viscosity silicone oils include, but are not limited to, Xiameter® silicone fluids from Dow Corning.

The at least one non-volatile silicone oil, if present, is preferably present in the compositions of the present invention in an amount ranging from about 2% to about 30% by weight, preferably from about 4% to about 25% by weight, and preferably from about 5% to about 20% by weight, based on the total weight of the composition, including all ranges and subranges within these ranges.

Colorants

The compositions of the present invention may optionally further comprise at least one colorant. Suitable colorants (coloring agents) include any colorant typically found in lip compositions. Suitable colorants include, but are not limited to, lipophilic dyes, pigments and pearlescent agents, and their mixtures.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

In accordance with preferred embodiments of lip compositions of the present invention, the colorant is preferably present in an amount sufficient to provide color to the lips, preferably in an amount of from about 0.1% to about 20% by weight, preferably from about 0.25% to about 15% by weight, and preferably from about 0.5 to about 10% by weight, of the total weight of the composition, including all ranges and subranges therebetween.

Auxiliaries/Additives

The compositions discussed above may additionally comprise an additive or auxiliary commonly used in cosmetic compositions and known to a person skilled in the art as being capable of being incorporated into a nail polish or varnish composition. Such additives or auxiliaries may be chosen from solvents, thickeners, coalescents, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

A person skilled in the art will take care to select the optional additional additives and/or the amount thereof such that the advantageous properties of the compositions according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the compositions of the invention should be cosmetically or dermatologically acceptable, i.e., they should contain a non-toxic physiologically acceptable. The compositions may be in any galenic form normally employed in the cosmetic and dermatological fields which is suitable for topical administration onto nails.

According to preferred embodiments of the present invention, methods of making a matte lip composition comprising combining at least one siloxysilicate resin in an amount of from about 7% to about 15% by weight, at least one polyorganosiloxane copolymer in an amount of from about 4.5% to about 10% by weight, at least one lipophilic silica in an amount of from about 0.5% to about 1.5% by weight, at least one non-lipophilic filler in an amount of from about 0% to about 3.5% by weight, and at least one non-volatile hydrocarbon oil in an amount of from about 0% to about 3.5% by weight, all weights being based on the total weight of the composition, during preparation of the lip composition to form a matte lip composition are provided.

According to preferred embodiments of the present invention, methods of making a matte lip composition comprising combining at least one siloxysilicate resin in an amount of from about 10% to about 14% by weight, at least one polyorganosiloxane copolymer in an amount of from about 6.5% to about 10% by weight, at least one lipophilic silica in an amount of from about 1% to about 1.5% by weight, at least one non-lipophilic filler in an amount of from about 1% to about 2% by weight, and at least one non-volatile hydrocarbon oil in an amount of from about 1% to about 2% by weight, all weights being based on the total weight of the composition, during preparation of the lip composition to form a matte lip composition are provided.

According to preferred embodiments of the present invention, methods of making up and/or protecting lips comprising applying to the lips a matte lip composition comprising at least one siloxysilicate resin in an amount of from about 7% to about 15% by weight, at least one polyorganosiloxane copolymer in an amount of from about 4.5% to about 10% by weight, at least one lipophilic silica in an amount of from about 0.5% to about 1.5% by weight, at least one non-lipophilic filler in an amount of from about 0% to about 3.5% by weight, and at least one non-volatile hydrocarbon oil in an amount of from about 0% to about 3.5% by weight, all weights being based on the total weight of the composition are provided.

According to preferred embodiments of the present invention, methods of making up and/or protecting lips comprising applying to the lips a matte lip composition comprising at least one siloxysilicate resin in an amount of from about 10% to about 14% by weight, at least one polyorganosiloxane copolymer in an amount of from about 6.5% to about 10% by weight, at least one lipophilic silica in an amount of from about 1% to about 1.5% by weight, at least one non-lipophilic filler in an amount of from about 1% to about 2% by weight, and at least one non-volatile hydrocarbon oil in an amount of from about 1% to about 2% by weight, all weights being based on the total weight of the composition are provided.

“Making up” as used herein means to provide decoration (for example, color) to the lips. “Protecting” as used herein means to inhibit damage to the lips such as, for example, sun damage.

In accordance with preferred embodiments of the preceding methods, at least one composition of the present invention is applied topically to the lips of a person in need of (desirous) the desired making up or protection in an amount sufficient to achieve the desired result. The compositions may be applied to the desired area as needed.

The compositions according to the invention can be manufactured by known processes used generally in the cosmetics or dermatological field.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

The present invention will be better understood from the examples which follow. The examples are intended to be nonrestrictive and explanatory only, with the scope of the invention defined by the claims.

### Example 1—Evaluation Techniques

Compositions were evaluated for tack, finish (matte), flake, according to the following procedures:

Tack: Films of compositions tested were deposited onto contrast cards using a 3 MIL drawdown bar and an Automatic Drawdown Machine. The films were dried at room temperature (25° C.) overnight and analyzed using a Texture Analyzer equipped with a ball probe. Tack force was measured after applying 350 g-force for 10 seconds. Then, the values of the tackiness were correlated to the comfort of wear of the tested products. The samples having tackiness values higher than 100 gr/force, were considered to be very uncomfortable to wear. The tack values between 50-100 gr/force, indicated medium comfort, and those with values of less than 50 gr/force were considered to be comfortable. In accordance with this testing, compositions of the present invention preferably have tack values of 50 gr/force or less, preferably 45 gr/force or less, preferably 35 gr/force or less, and preferably 25 gr/force or less.

In vitro Shine: Films of compositions tested were deposited onto BLACK SCRUB PANEL P121-10N using a 1 MIL drawdown bar and an Automatic Drawdown Machine. The films were dried at room temperature for 5 minutes and analyzed using a gloss meter (BYK: micro-TRI-gloss) at an angle of 60°.

Flake: Samples were tested for flake resistant properties. Samples of all tested compositions were deposited onto polyurethane white bioskin using a 3 MIL drawdown bar. The samples were allowed to dry overnight at room temperature (25° C.) and then they were stretched by pulling from two sides. During the stretching process, flaking of the dry samples was observed and correlated with durability of wear of the tested products. To define the degree of samples' flaking, the following four (5) point grading scale was used: 1—no flaking (very good wear), 2—low flaking (good wear), 3—medium flaking (medium/acceptable wear), 4—high flaking (no wear) and 5—fail (no wear).

Separation (lack of stability): Compositions were hand mixed and 6 mL transferred into a 15 mL plastic centrifuge tube. The samples were centrifuged for 25 minutes at 2800G (Themo Electron Corporation IEC C130 Centrifuge). Percent separation was calculated using the equation % separation=[(XmL separated transparent liquid)/6 mL]×100.

### Example 2—Composition Preparation

Compositions were prepared by the following procedure: Pigment, isododecane and MQ resin were ground to create a pigment paste. The paste was processed using Disconti Mill until it passed the Hegman Gauge test (ASTM D1210-05). Then, the paste was added to the remaining ingredients. The mixture was heated to 80° C. and stirred, until a homogeneous liquid composition was obtained. After that, the composition was cooled down to the room temperature and transferred to desired containers and/or applicators.

### Example 3—Comparative Testing

Different amounts of siloxysilicate resin (trimethylsiloxysilicate), polyorganosiloxane copolymer (nylon 611/dimethicone copolymer), lipophilic silica (silica silylate), non-lipophilic filler, and non-volatile hydrocarbon oil (polybutene) were evaluated in a base lip composition to prepare both invention and comparative compositions. The variations and results associated with them are provided below:

Invention Compositions

Pigment grind

| | | | | | | |
|---|---|---|---|---|---|---|
| TRIMETHYLSILOXYSILICATE | 3 | 3 | 3 | 3 | 3 | 3 |
| ISODODECANE | 17 | 17 | 17 | 17 | 17 | 17 |
| LAUROYL LYSINE | 1.5 | 1.5 | 1.5 | 1.5 | 1.5 | 1.5 |
| Pigment | 10 | 10 | 10 | 10 | 10 | 10 |
| Invention Compositions | A | B | C | D | E | F |
| Pigment grind | 31.5 | 31.5 | 31.5 | 31.5 | 31.5 | 31.5 |
| Bentone Gel (10% Active) | 25 | 25 | 25 | 25 | 25 | 25 |
| Silicone resin wax | 0.5 | 0.5 | 0.5 | 0.5 | 0.5 | 0.5 |
| TRIMETHYLSILOXYSILICATE | 12 | 12 | 10.8 | 4 | 9 | 7.3 |
| (additional) | | | | | | |
| NYLON-611/DIMETHICONE | 10 | 10 | 9.2 | 4.7 | 8 | 6.8 |
| COPOLYMER | | | | | | |
| POLYBUTENE (605-655 cSt at 100 C.) | 3.3 | 3.3 | 3.5 | 3.3 | 3.5 | 2.4 |
| SILICA SILYLATE | 0.5 | 0.5 | 1.4 | 1.5 | 1.2 | 1.2 |
| NYLON-12 | 0 | 2.5 | 1.7 | 5 | 1.7 | 1.3 |
| ISODODECANE | QS | QS | QS | QS | QS | QS |
| Tack | 65.7 | 56.7 | 44.8 | 2.8 | 33.9 | 15.2 |
| Flake | 1.0 | 1.0 | 1.0 | 3.0 | 2.0 | 3.0 |
| % Separation | 0.0 | 0.0 | 0.0 | 0.0 | 0.0 | 0.0 |
| Shine | 0.1 | 0.1 | 0.1 | 0.1 | 0.1 | 0.1 |

Comparative Compositions

Pigment grind

| | | | | | | |
|---|---|---|---|---|---|---|
| TRIMETHYLSILOXYSILICATE | 3 | 3 | 3 | 3 | | |
| ISODODECANE | 17 | 17 | 17 | 17 | | |
| LAUROYL LYSINE | 1.5 | 1.5 | 1.5 | 1.5 | | |
| Pigment | 10 | 10 | 10 | 10 | | |
| Comparative Compositions | 1 | 2 | 3 | 4 | 5* | 6** |
| Pigment grind | 31.5 | 31.5 | 31.5 | 31.5 | | |
| Bentone Gel (10% Active) | 25 | 25 | 25 | 25 | | |
| Silicone resin wax | 0.5 | 0.5 | 0.5 | 0.5 | | |
| TRIMETHYLSILOXYSILICATE | 0 | 0 | 8 | 4 | | |
| (additional) | | | | | | |
| NYLON-611/DIMETHICONE | 2 | 2 | 7.3 | 4.7 | | |
| COPOLYMER | | | | | | |
| POLYBUTENE | 0 | 3.3 | 10 | 0 | | |
| SILICA SILYLATE | 0 | 0.5 | 0.5 | 0 | | |
| NYLON-12 | 0 | 0 | 5 | 5 | | |
| ISODODECANE | QS | QS | QS | QS | | |
| Tack | 0.3 | 2.1 | 26.3 | 0.3 | 92.6 | 10.5 |
| Flake | 4.0 | 4.0 | 2.0 | 3.0 | 1.0 | 4.0 |
| % Separation | 42.9 | 40.5 | 15.4 | 7.1 | 0.0 | |
| Shine | 0.1 | 0.1 | 0.1 | 0.1 | 5.7 | 0.1 |

Comparative Composition 5 corresponds to a commercial product containing the following ingredients:

Isododecane, trimethylsiloxysilicate, nylon-611/dimethicone copolymer, disteardimonium hectorite, lauroyl lysine, c30-45 alkyldimethylsilyl polypropylsilsesquioxane, alumina, propylene carbonate, synthetic fluorphlogopite, silica, calcium sodium borosilicate, calcium aluminum borosilicate, polyethylene terephthalate, partum/fragrance, aluminum hydroxide, acrylates copolymer, benzyl alcohol, dimethicone, paraffin, tin oxide. [+/− may contain/peut contenir mica, ci 77891/titanium dioxide, ci 77491, ci 77492, ci 77499/iron oxides, ci 15850/red 7, ci 15985/yellow 6 lake, ci 45410/red 28 lake, ci 45380/red 22 lake, ci 19140/yellow 5 lake, ci 42090/blue 1 lake, ci 75470/carmine]. 111. d41008/5

**Comparative Composition 6 corresponds to a commercial product containing the following ingredients:

Isododecane, Dimethicone, Trimethylsiloxysilicate, Polybutene, Petrolatum, Cyclohexasiloxane, Kaolin, Disteardimonium Hectorite, Beeswax/Cera Alba/Cire D'abeille, Silica Dimethyl Silylate, Tocopherol, Persea Graissima(Avocado)Oil, Cyclopentasiloxane, Glyceryl Behenate/Eicosadioate, Tin Oxide, Propylene Carbonate, Caprylyl Glycol, Hexylene Glycol, Flavor(Aroma), Phenoxyethanol. MAY CONTAIN/PEUTCONTENIR (+/−): TITANIUM DIOXIDE, IRON OXIDES, RED 7 LAKE, RED 27 LAKE, BLUE 1

1. A matte lip composition comprising at least one siloxysilicate resin in an amount of from about 7% to about 15% by weight, at least one polyorganosiloxane copolymer in an amount of from about 4.5% to about 10% by weight, at least one lipophilic silica in an amount of from about 0.5% to about 1.5% by weight, at least one non-lipophilic filler in an amount of greater than 0% to about 3.5% by weight, and at least one non-volatile hydrocarbon oil in an amount of greater than 0% to about 3.5% by weight, all weights being based on the total weight of the composition.

2. The matte lip composition of claim 1, further comprising at least one colorant and at least one volatile solvent.

3. The matte lip composition of claim 1, wherein the composition has average gloss properties, measured at 60°, of less than or equal to 5.

4. The matte lip composition of claim 1, wherein the composition has average gloss properties, measured at 60°, of less than or equal to 1.

5. The matte lip composition of claim 1, wherein the siloxysilicate resin is trimethylsiloxysilicate.

6. The matte lip composition of claim 1, wherein the polyorganosiloxane copolymer is nylon-611/dimethicone.

7. The matte lip composition of claim 1, wherein the lipophilic silica is silica silylate.

8. The matte lip composition of claim 1, wherein the non-lipophilic filler is a polyamide powder comprising particles having an average particle size ranging from 5 to 60 microns, having a specific surface area ranging from 1 to 20 m²/g, having a density ranging from 1.03 to 1.15 g/cm³, and having a melting point ranging from 140° C. to 220° C.

9. The matte lip composition of claim 1, wherein the non-volatile hydrocarbon oil is a C3-C5 polyalkene.

10. The matte lip composition of claim 1, wherein the non-volatile hydrocarbon oil is polybutene.

11. A method of making up lips comprising applying the composition of claim 1 to the lips.

12. A matte lip composition comprising at least one siloxysilicate resin in an amount of from about 10% to about 14% by weight, at least one nylon-611/dimethicone in an amount of from about 6.5% to about 10% by weight, at least one lipophilic silica in an amount of from about 1% to about 1.5% by weight, at least one polyamide powder in an amount of from about 1% to about 2% by weight, and at least one C3-C5 alkene in an amount of from about 1% to about 2% by weight, all weights being based on the total weight of the composition, wherein the composition has average gloss properties, measured at 60°, of less than or equal to 5.

13. The matte lip composition of claim 12, further comprising at least one colorant and at least one volatile solvent.

14. (canceled)

15. The matte lip composition of claim 12, wherein the composition has average gloss properties, measured at 60°, of less than or equal to 1.

16. The matte lip composition of claim 12, wherein the siloxysilicate resin is trimethylsiloxysilicate.

17. (canceled)

18. The matte lip composition of claim 12, wherein the lipophilic silica is silica silylate.

19. The matte lip composition of claim 12, wherein the polyamide powder comprises particles having an average particle size ranging from 5 to 60 microns, having a specific surface area ranging from 1 to 20 m²/g, having a density ranging from 1.03 to 1.15 g/cm³, and having a melting point ranging from 140° C. to 220° C.

20. The matte lip composition of claim 12, wherein the C3-C5 polyalkene is polybutene.
